(19) United States
(12) Patent Application Publication
(10) Pub. No.: US 2004/0147954 A1
(43) Pub. Date: Jul. 29, 2004

(54) Internal nasal dilator

(76) Inventor: Charles E. Wood, Delray Beach, FL (US)

Correspondence Address: Mark D. Bowen, Stearns Weaver Miller, et al., Suite 1900, 200 East Broward Boulevard, Fort Lauderdale, FL, 33301, US

(21) Appl. No.: 10/757,117

(22) Filed: Jan. 14, 2004

Related U.S. Application Data
(60) Provisional application No. 60/439,850, filed on Jan. 14, 2003.

(51) Int. Cl. A61M 29/00

(52) U.S. Cl. 606/199

(57) ABSTRACT

An internal nasal dilator sized for comfortable insertion within the human nostril and adapted with integral particulate filtration media is disclosed. The nasal dilator is adapted to house air filtration technology such that air drawn through the device is filtered prior to entering the lungs. Air filtration may be accomplished using particulate filter media, chemical media (such as activated carbon), centrifugal particulate separation technology, or any other suitable air filtration technology. The present invention thus provides an improved internal nasal dilator which functions to maintain clear and unobstructed nasal passages while improving the quality of breathing air by removing particulate matter prior to introduction into the lungs.

### CROSS REFERENCE TO RELATED APPLICATIONS

This application claims the benefit of U.S. Provisional Patent Application Serial No. 60/439,850, filed Jan. 14, 2003.

### STATEMENT REGARDING FEDERALLY SPONSORED RESEARCH OR DEVELOPMENT

N/A

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office patent file or records, but otherwise reserves all copyrights.

### BACKGROUND OF THE INVENTION

1. Field of the Invention

This invention relates to nasal dilators, and, more particularly to internal nasal dilators adapted for insertion in the nostril of the human nose to provide improved breathing capability and breathing air quality.

2. Description of the Background Art

A large percentage of the human population is afflicted with nasal malformations that impede breathing capability. In addition, breathing is often impeded when nasal passages restrict during inhalation. The problem is most pronounced during periods of strenuous exercise where respiration increases and maximum volumes of air are drawn in through the nasal passages to provide an increased supply of oxygen.

The anatomical structure of the human nose contributes to the difficulties experienced when breathing. Specifically, the lower internal portion of each nostril, known as the vestibule, typically tapers inward to a narrowed area, known as the ostium internum, which defines the nasal air passage leading to the lungs. The nasal wall defining the vestibule is prone to being drawn in during inhalation to the point where airflow through the ostium internum is restricted.

In response to this problem, others have proposed the use of nasal dilators to provide unobstructed nasal passages. The following patents provide examples of both internal and external nasal dilators which function as nostril propping devices and inhalation aids.

| | |
|---|---|
| 4,414,977 | Rezakhany |
| 5,533,499 | Johnson |
| 5,706,800 | Cronk et al. |
| 5,816,241 | Cook |
| 5,895,409 | Mehdizadeh |
| 5,961,537 | Gould |
| 6,006,746 | Karell |
| 6,080,179 | Gould |
| 6,106,541 | Hurbis |
| 6,238,411 | Thorner |
| 6,270,512 | Rittmann |
| 6,276,360 | Cronk et al. |
| 6,318,362 | Johnson |
| 6,328,754 | Marten et al. |
| Des. 388,172 | Cipes |
| Des. 422,703 | Lundy, Jr. et al. |
| Des. 430,667 | Rome |

The references of the background art generally disclose two types of inhalation aids, namely, internal and external nasal dilators. Nasal dilators function to provide improved breathing capability for persons by expanding the cross sectional area of the nasal passage and thereby increasing the capacity of gas (e.g. air, oxygen etc.) inhalation. External nasal dilators are characterized by strips that are typically adhesively secured to the nose and function externally to prevent the nasal passages from drawing in during inhalation. External nasal dilators are shown in U.S. Pat. No. 5,533,499 (Johnson), U.S. Pat. No. 5,706,800 and U.S. Pat. No. 6,276,360 (Cronk et al.), U.S. Pat. No. 6,006,746 (Karell), U.S. Pat. No. 6,080,179 (Gould), U.S. Pat. No. 6,238,411 (Thorner), and U.S. Pat. No. 6,318,362 (Johnson). Internal nasal dilators are characterized by structures designed for insertion into the nostrils and function internally to prevent the nasal passages from drawing in during inhalation. Examples of internal nasal dilators are disclosed in U.S. Pat. No. 4,414,977 (Rezakhany), U.S. Pat. No. 5,816,241 (Cook), U.S. Pat. No. 5,895,409 (Mehdizadeh), U.S. Pat. No. 6,106,541 (Hurbis), U.S. Pat. No. 6,270,512 (Rittmann), and Des. 430,667.

U.S. Pat. No. 5,895,409 (the “'409 patent”), issued to Mehdizadeh, discloses an internal nasal dilator designed for insertion within the human nostril. The device comprises an open framework with no internal members, that, upon insertion, prevents the collapse of the internal nasal wall. The various embodiments disclosed by Mehdizadeh, include various frame configurations having dimensions that are sufficient to comfortably seat within the human nostril.

While the devices disclosed are generally suitable for their intended purposes, the devices suffer from a number of significant shortcomings and disadvantages. More particularly, none of the nasal dilators disclosed in the art improve breathing capability by removing particulate matter and airborne debris from the breathing air. In addition, it is noted that internal nasal dilators disclosed in the background art actually interfere with the ability of the nostril hairs to filter particulate matter. Even internal nasal dilators having open framework structures interfere with nostril filtration by disturbing and/or interfering with the matrix of hair projecting within the nostrils from the internal nasal wall. Accordingly, the background art fails to provide an internal nasal dilator that improves breathing capability by maintaining open nasal passages while improving the quality of breathing air by removing particulate matter and airborne debris.

### BRIEF SUMMARY OF THE INVENTION

The present invention addresses and overcomes the disadvantages and shortcomings in the art by providing an internal nasal dilator adapted with integral particulate filtration media. In a preferred embodiment, the present invention comprises an internal nasal dilator having an open framework structure sized for comfortable insertion within the human nostril. The framework is adapted to house air filtration technology such that air drawn through the device is filtered prior to entering the lungs. Air filtration may be accomplished using particulate filter media, chemical media (such as activated carbon), centrifugal particulate separation technology, or any other suitable air filtration technology. The present invention thus provides an improved internal nasal dilator which functions to maintain clear and unobstructed nasal passages while improving the quality of breathing air by removing particulate matter prior to introduction into the lungs.

Accordingly, it is an object of the invention to provide an improved nasal dilator.

Still another object of the invention is to provide an internal nasal dilator adapted to filter the air drawn through the device.

Yet another object of the present invention is to provide an internal nasal dilator adapted to include airborne particulate filter media.

Another object of the present invention is to provide an internal nasal dilator adapted to include chemical air filtration media.

Still another object of the present invention is to provide an internal nasal dilator adapted to include centrifugal particulate filtration.

Still further objects and advantages will become apparent from a consideration of the ensuing description and drawings.

### BRIEF DESCRIPTION OF THE SEVERAL VIEWS OF THE DRAWINGS

FIG. 1 depicts an internal nasal dilator disclosed in the background art;

FIG. 2 depicts an exploded top perspective view of an internal nasal dilator according to the present invention;

FIG. 3 depicts an exploded bottom perspective view thereof;

FIG. 4 depicts a top perspective view thereof;

FIG. 5 depicts a partial side sectional view thereof;

FIG. 6 illustrates a side view of an internal nasal dilator operatively inserted within a human nose;

FIG. 7 illustrates a front view of an internal nasal dilator operatively inserted within a human nose;

FIG. 8 depicts an alternate embodiment single nostril internal nasal dilator;

FIG. 9 is a cross-sectional view thereof depicting an alternate embodiment air filtration media;

FIG. 10 depicts a dual nostril embodiment thereof;

FIG. 11 depicts an alternate embodiment formed of woven material;

FIG. 12 depicts an alternate embodiment incorporating dual filtration disks;

FIG. 13 depicts an alternate embodiment incorporating synthetic nostril hair; and

FIG. 14 depicts an alternate embodiment incorporating a replaceable conical filter.

### DETAILED DESCRIPTION OF THE INVENTION

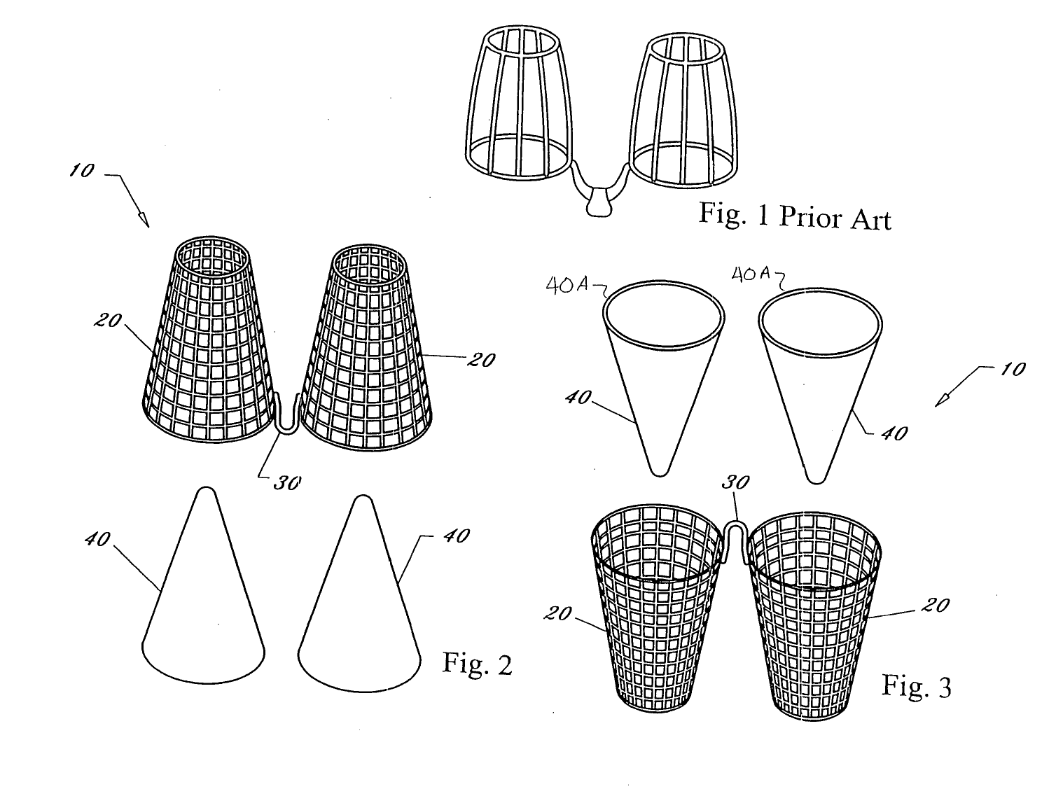

With reference to the drawings, FIG. 1 illustrates a prior art internal nasal dilator disclosed in the background art. The nasal dilator shown in FIG. 1 is characterized by an open framework structure having left and right nostril modules sized for comfortable insertion within the human nasal passages. Devices similar to the internal nasal dilator depicted in FIG. 1 are sized for comfortable insertion within the human nose such that the left and right nostril modules function to maintain clear nasal passages by propping open the internal nasal walls. As a result, breathing is facilitated by preventing the collapse of the nasal walls during inhalation.

FIGS. 2-5 depict an internal nasal dilator, generally referenced as 10, in accordance with the present invention. Nasal dilator 10 includes at least one, and preferably two, tubular mesh nostril insert structures, each of which is generally referenced as 20, sized and shaped for removable insertion within the human nostril(s). In a preferred embodiment, nasal dilator 10 includes a pair of similarly shaped nostril insert structures that are connectably attached by a cross member 30. Cross member 30 functions to assist the user with comfortable and selective insertion and removal of nasal dilator 10.

Each nostril insert 20 is preferably fabricated from a soft and resilient material configured in tubular mesh form sized and shaped for removable insertion within the nostril. Nostril inserts 20 are preferably defined by tubular structures formed by interwoven mesh of resilient elongate members. The tubular mesh configuration provides superior internal support to the nasal wall structure due to the superior support structure inherent in an interwoven configuration. Another advantage realized by the mesh structure of the present invention relates to retaining the filtration functionality of the nasal hair structures of the human nose. More particularly, the openings in the mesh allow nasal hair structures to retain functionality by projecting through the mesh. Thus, the nasal hair structures retain the ability to filter particulate matter from the air drawn in through the device.

A significant aspect of the present invention relates to breathing air quality. More particularly, the nasal dilator of the present invention is specifically adapted with integral air filtration technology, generally referenced as 40, to remove particulate and/or chemical contaminants from the air. The air filtration technology is preferably disposed within the void defined by the tubular structure forming each nostril insert 20 such that air drawn in through each nostril insert is forced through the air filtration technology.

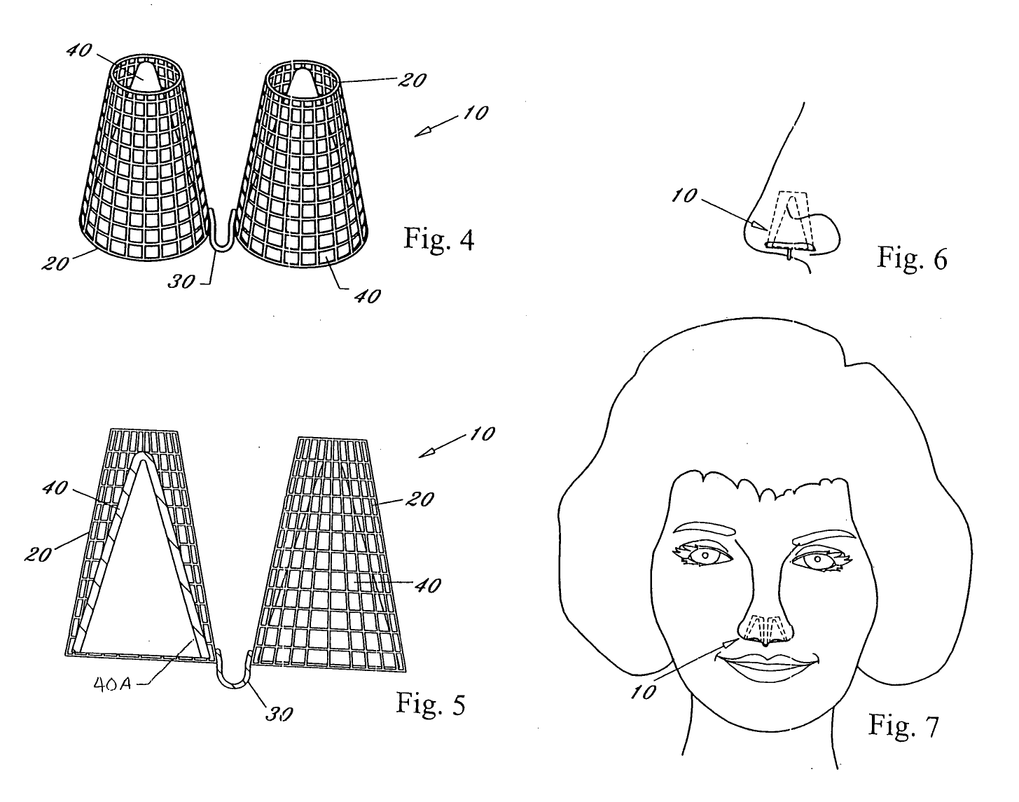

In the preferred embodiment depicted in FIGS. 3-5, the air filtration technology 40 comprises particulate air filtration media. The particulate air filtration media is preferably sized and shaped for removable insertion within nostril inserts 20 as shown in FIGS. 2-5. As should be apparent, the air filtration media is disposed such that air drawn through the device is filtered prior to entering the lungs. In the embodiment depicted in FIGS. 2-5, air filtration is accomplished by particulate air filtration media configured in a hollow conical shape, referenced as 40. The conical filters are removably inserted within each nostril insert 20 as illustrated in FIGS. 4 and 5. Accordingly, when the internal nasal dilator 10 is inserted within the user's nostrils as shown in FIGS. 6 and 7, air drawn in through the nasal passages enters the open end 40a of air filters 40 and passes through the filter wall prior to entering the user's lungs. The hollow conical filter configuration provides relatively large filter surface area that facilitates air filtration at a minimum resistance. Providing air filtration technology that removes particulate contamination while minimizing resistance to air flow is considered a significant aspect of the present invention.

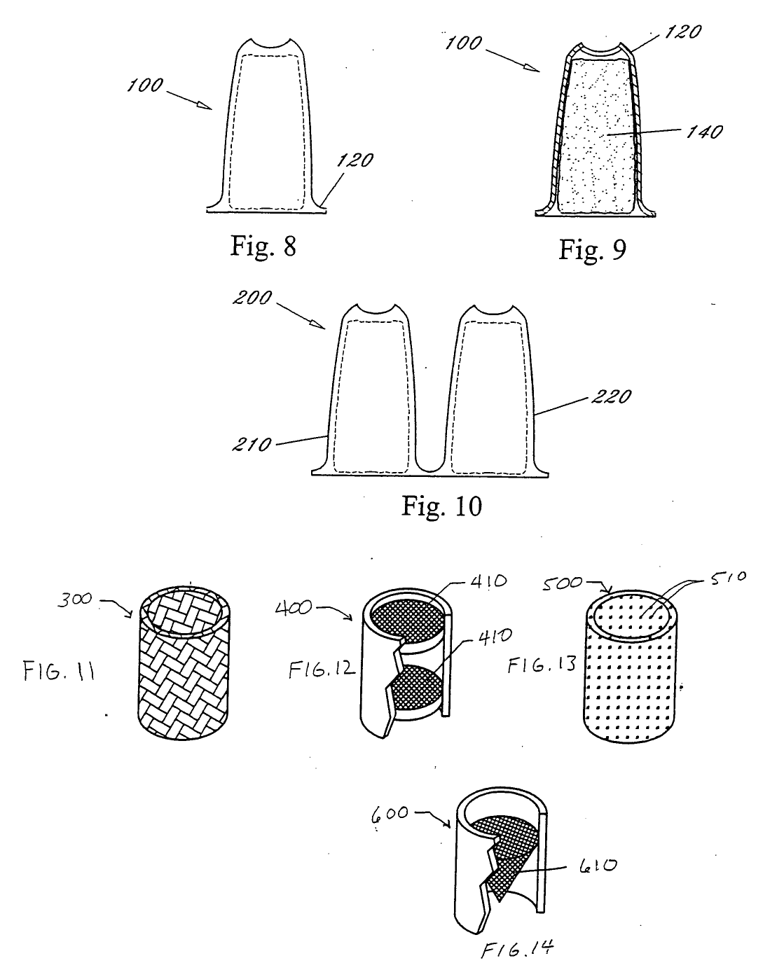

FIG. 8 depicts an alternate embodiment single nostril internal nasal dilator referenced as 100. Nasal dilator 100 comprises a uniform tubular body 120 having open ends and sized for nostril insertion. FIG. 9 is a cross-sectional view of nasal dilator 100 adapted with an alternate embodiment air filtration media, referenced as 140. Air filtration media 140 is preferably fibrous wad of cotton or other suitable fiber material. The cotton fiber has been found to provide adequate air filtration characteristics with minimum restriction, e.g. pressure drop across the filter media, thereby maintaining breathability. FIG. 10 depicts a dual nostril internal nasal dilator embodiment, generally referenced as 200. Nasal dilator 200 includes left and right nostril inserts, referenced as 210 and 220 respectively, each of which may have filter media insertably received therein as generally illustrated in FIGS. 8 and 9.

FIG. 11 depicts an alternate embodiment internal nasal dilator 300 formed of woven material to facilitate insertion into the nasal passage. The use of woven material results in a nasal dilator that adapts in size to fit snugly within the nasal passage. FIG. 12 depicts an alternate embodiment internal nasal dilator 400 incorporating dual filtration disks, each referenced as 410. Filtration disks 410 are preferably replaceable and disposable. In one embodiment, filtration disks 410 may include a pre-filter and primary filter each having different filtering characteristics. FIG. 13 depicts an alternate embodiment internal nasal dilator 500 incorporating synthetic nostril hair members, referenced as 510, projecting radially inward. FIG. 14 depicts an alternate embodiment internal nasal dilator 600 incorporating a replaceable inverted conical filter 610. Conical filter 610 is preferably replaceable and disposable. It should be noted that each of the various embodiments depicted in FIGS. 11-14 may be used as individual units or may be configured in pairs in an attached configuration similar to that shown in FIG. 10.

In various alternate embodiments, air filtration may be accomplished using particulate filter media, chemical media, or centrifugal separation technology. In addition, the particulate filter media may be impregnated with activated carbon to assist in odor removal. The present invention thus provides an improved internal nasal dilator which functions to maintain clear and unobstructed nasal passages while improving the quality of breathing air by removing particulate matter prior to introduction into the lungs.

The instant invention has been shown and described herein in what is considered to be the most practical and preferred embodiment. It is recognized, however, that departures may be made therefrom within the scope of the invention and that obvious structural and/or functional modifications will occur to a person skilled in the art.

What I claim is:

1. An internal nasal dilator for insertion within a single nostril comprising:
a generally cylindrical nasal passage dilator having a tapered body defining open top and bottom ends and an interior volume;
a filter insertably disposed within said interior volume defined by said tapered body of said nasal passage dilator.

2. An internal nasal dilator according to claim 1, wherein said tapered body is formed from a plurality of interconnected elongate tubular members.

3. An internal nasal dilator according to claim 1, wherein said tapered body is formed from a rubber-like material.

4. An internal nasal dilator according to claim 1, wherein said filter comprises a hollow, conically-shaped particulate filter sized for insertion within said interior volume defined by said tapered body of said nasal passage dilator.

5. An internal nasal dilator according to claim 1, wherein said filter comprises a wad of cotton material.

6. An internal nasal dilator according to claim 1, wherein said filter comprises a chemical media.

7. An internal nasal dilator according to claim 6, wherein said chemical media includes activated carbon.

8. An internal nasal dilator for insertion within a person's nostrils, said internal nasal dilator comprising:
a pair of generally cylindrical nasal passage dilators, each of said nasal passage dilators having a tapered body defining open top and bottom ends and an interior volume;
a generally U-shaped connecting link extending between said tapered body bottom ends;
a filter insertably disposed within the interior volume defined by said tapered body of each of said nasal passage dilators.

9. An internal nasal dilator according to claim 8, wherein said filter is a particulate filter.

10. An internal nasal dilator according to claim 8, wherein said particulate filter includes first and second stages of filtration.

11. An internal nasal dilator according to claim 10, wherein said first stage of filtration comprises a particulate filter and said second stage of filtration comprises a chemical media.
